(19) United States
(12) Patent Application Publication
(10) Pub. No.: US 2025/0161242 A1
(43) Pub. Date: May 22, 2025

(54) TRICHOSTATIN A (TSA) SENSITIVITY IN THE TREATMENT OF TUMORS

(71) Applicant: VANDA PHARMACEUTICALS INC., Washington, DC (US)

(72) Inventors: Bartlomiej PRZYCHODZEN, Washington, DC (US); Mihael POLYMEROPOULOS, Washington, DC (US)

(21) Appl. No.: 18/839,564

(22) PCT Filed: Feb. 27, 2023
(86) PCT No.: PCT/US2023/063329

§ 371 Date: Aug. 19, 2024

Related U.S. Application Data
(60) Provisional application No. 63/268,714, filed on Mar. 1, 2022.

(51) Int. Cl. A61K 31/165; C12Q 1/6886

(52) U.S. Cl. CPC A61K 31/165; C12Q 1/6886; C12Q 2600/106; C12Q 2600/158

(57) ABSTRACT

Embodiments of the invention relate generally to the treatment of tumors and, more particularly, to the use of trichostatin A (TSA) in tumor treatment.

### CROSS-REFERENCE TO RELATED APPLICATIONS

This application claims the benefit of co-pending U.S. Provisional Patent Application Ser. No. 63/268,714, filed 1 Mar. 2022, which is hereby incorporated herein as though fully set forth.

### BACKGROUND

Histone deacetylase inhibitors (HDACis) are small molecules that increase acetylation of lysine residues by blocking histone deacetylases (HDACs). HDACis affect epigenetic and non-epigenetic gene expression, resulting in cell cycle arrest of cancer cells and can also enhance anti-tumor effects via the pharmacologic modulation of the suppressive activity of macrophages. Some HDACis, such as trichostatin A (TSA), can also affect the tumor immune microenvironment by suppressing the activity of infiltrating macrophages and inhibiting myeloid-derived suppressor cell (MDSC) recruitment. Trichostatin A, the compound of Formula I below, is further known as [R-(E,E)]-7-[4-(dimethylamino)phenyl]-N-hydroxy-4,6-dimethyl-7-oxo-2,4-heptadienamide.

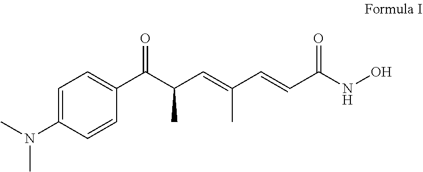 [SMILES: CC(/C=C/C(=O)NO)=C\[C@@H](C)C(=O)c1ccc(N(C)C)cc1]

### SUMMARY

Embodiments of the invention relate generally to the suppression of tumors and, more particularly, to the use of trichostatin A (TSA) in the suppression of tumors in cancer patients determined to be sensitive to TSA treatment. Cancer patients, as referenced herein, refers to individuals diagnosed with a malignancy for which TSA has been described as a possible therapeutic option, including patients as described in U.S. Pat. No. 10,265,282, e.g., patients diagnosed with breast cancer, gastric cancer, colon cancer, rectal cancer, bladder cancer, pancreatic cancer, ovarian cancer, prostate cancer, lung cancer, hematological cancer, and skin cancer.

In one embodiment, the invention provides, in a method of administering trichostatin A (TSA) to a cancer patient, an improvement comprising: selecting as said patient an individual identified as overexpressing at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP.

In another embodiment, the invention provides, in a method of administering trichostatin A (TSA) to a cancer patient, an improvement comprising: selecting as said patient an individual identified as under-expressing at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36.

In yet another embodiment, the invention provides a method of inhibiting tumor growth in a cancer patient, the method comprising: determining or having determined that the patient exhibits: increased expression of at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP; or decreased expression of at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36; and administering to the patient trichostatin A (TSA) in an amount effective to inhibit tumor growth.

In still another embodiment, the invention provides a method of enhancing a tumor suppression therapy in which a cancer patient is administered a tumor suppression composition, the method comprising: determining or having determined that the patient exhibits: increased expression of at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP; or decreased expression of at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36; and administering to the patient trichostatin A (TSA) in an amount effective to inhibit tumor growth.

For cancer patients being treated in accordance with the above improvement, the therapeutic regimen for treatment follows procedures described in the art for cancer patients receiving TSA therapy. TSA may be administered to the individual to be treated in the form of a pharmaceutical composition. Pharmaceutical compositions to be used comprise a therapeutically effective amount of TSA (i.e., either TSA itself or a pharmaceutically acceptable salt or other form, such as a solvate thereof), together with one or more pharmaceutically acceptable excipients or carriers. As known in the art, administration may be oral but other routes of administration may also be employed, e.g., parenteral, nasal, buccal, transdermal, sublingual, intramuscular, intravenous, rectal, and vaginal. Solid dosage forms for oral administration include capsules, tablets, pills, powders, and granules. In such solid dosage forms, the compound is admixed with at least one inert pharmaceutically acceptable excipient.

Upon determining that a cancer patient exhibits such greater-than-normal expression and/or lesser-than-normal expression, the patient may be administered TSA in an amount effective to inhibit tumor growth. For example, an amount of TSA effective to inhibit tumor growth may be an amount between about 0.1 mg/kg/day and about 10 mg/kg/day, e.g., between about 0.5 mg/kg/day and about 5 mg/kg/day. The actual dose of TSA administered to a patient may depend, for example, on the patient's age, weight, severity of disease, co-morbidities, and other factors known in the art. Otherwise, the dosage forms, dosage amounts, and routes of administration for a particular patient are determined using conventional dose-finding and dosing methodologies as known in the pharmaceutical and medical arts. In addition, TSA administration may supplement the administration of other oncolytic agents, including tumor suppressing compounds.

### DETAILED DESCRIPTION

Some hematological malignancies are known to exhibit greater or lesser sensitivity to treatment with TSA. A high-throughput screening of gene expression profiles in known malignant hematological cell lines reveals transcriptional signatures associated with both TSA sensitivity and TSA resistance. As used herein, TSA sensitivity means a relative difference in expression between top 10^{th} percentile of most sensitive cell lines and expression of the 10^{th} percentile of most resistant cell lines. Higher or lower expression shall mean an increase or decrease in a level of expression in malignant cell lines exhibiting TSA sensitivity.

Within malignant cell lines exhibiting TSA sensitivity, the expression of 49 genes is upregulated at least two-fold among most sensitive cell line models, while the expression of 85 genes is downregulated at least two-fold in response to the same range of concentration tested. Data related to the upregulated genes are shown in Table 1. Data related to the downregulated genes are shown in Table 2. In each, sensitive and resistant measures are in fragments per kilobase of exon per million mapped fragments (FPKM).

TABLE 1
genes upregulated in cell lines exhibiting TSA sensitivity

| | Sens. | Resist. | | |
|---|---|---|---|---|
| GENE | (FPKM) | (FPKM) | s/r | p |
| TLE4 | 78.84 | 39.36 | 2.00 | 0.001009 |
| SMO | 17.97 | 8.95 | 2.01 | 0.039308 |
| GPC2 | 19.39 | 9.59 | 2.02 | 0.003264 |
| SNTA1 | 22.11 | 10.79 | 2.05 | 0.016975 |
| YPEL1 | 29.29 | 14.28 | 2.05 | 0.003575 |
| PLEKHA2 | 260.63 | 125.94 | 2.07 | 0.000194 |
| GPR137C | 5.47 | 2.64 | 2.08 | 0.001513 |
| CD72 | 46.72 | 22.48 | 2.08 | 0.044525 |
| ZFP36L1 | 245.89 | 117.82 | 2.09 | 0.005234 |
| PAX5 | 462.79 | 220.71 | 2.10 | 0.008790 |
| ZCCHC7 | 184.16 | 85.04 | 2.17 | 0.000139 |
| ADARB1 | 95.59 | 43.81 | 2.18 | 0.001015 |
| SMIM14 | 101.16 | 46.09 | 2.19 | 0.018359 |
| SNX22 | 42.17 | 18.82 | 2.24 | 0.009975 |
| AFF2 | 68.35 | 30.47 | 2.24 | 0.011847 |
| GJC1 | 57.12 | 25.45 | 2.24 | 0.001664 |
| RIPOR2 | 134.67 | 59.01 | 2.28 | 0.000940 |
| ARID5B | 86.92 | 38.05 | 2.28 | 0.000332 |
| RAB9B | 5.53 | 2.41 | 2.29 | 0.002421 |
| MIR570HG | 59.56 | 25.94 | 2.30 | 0.001112 |
| CD79B | 344.38 | 149.41 | 2.30 | 0.006246 |
| IKZF2 | 240.49 | 104.28 | 2.31 | 0.004045 |
| ZNF318 | 232.19 | 97.41 | 2.38 | 0.000130 |
| EBF1 | 108.57 | 45.08 | 2.41 | 0.008646 |
| SOX4 | 366.48 | 150.87 | 2.43 | 0.001139 |
| TRIB2 | 137.13 | 56.45 | 2.43 | 0.001527 |
| GRAP | 39.87 | 16.30 | 2.45 | 0.004641 |
| TTC24 | 16.93 | 6.84 | 2.48 | 0.005468 |
| RASGRP1 | 91.79 | 37.04 | 2.48 | 0.003404 |
| MIR5195 | 6.05 | 2.43 | 2.50 | 0.018490 |
| FAM81A | 23.13 | 9.16 | 2.53 | 0.009647 |
| IGHV5-78 | 14.19 | 5.61 | 2.53 | 0.031847 |
| LRMP | 763.67 | 292.66 | 2.61 | 0.000582 |
| MS4A1 | 736.42 | 268.81 | 2.74 | 0.004268 |
| GPR146 | 8.41 | 2.97 | 2.83 | 0.048580 |
| CHI3L2 | 251.51 | 88.52 | 2.84 | 0.035511 |
| ZNF608 | 130.81 | 45.47 | 2.88 | 0.001153 |
| CDC25B | 348.49 | 114.37 | 3.05 | 0.000116 |
| C12orf77 | 11.96 | 3.85 | 3.11 | 0.001756 |
| GPR18 | 29.04 | 9.33 | 3.11 | 0.000656 |
| PCDH12 | 8.43 | 2.62 | 3.22 | 0.042406 |
| SCML4 | 5.31 | 1.58 | 3.36 | 0.021971 |
| CD24 | 386.36 | 112.91 | 3.42 | 0.000557 |
| INA | 9.55 | 2.73 | 3.50 | 0.011007 |
| TUNAR | 22.87 | 5.70 | 4.01 | 0.046206 |
| FAM241B | 6.28 | 1.55 | 4.05 | 0.010487 |
| TCL1B | 25.88 | 3.35 | 7.73 | 0.002416 |
| IGLV1-44 | 108.92 | 9.01 | 12.09 | 0.013573 |
| ENPEP | 50.04 | 3.47 | 14.41 | 0.007325 |

TABLE 2
genes downregulated in cell lines exhibiting TSA sensitivity

| | Sens. | Resist. | | |
|---|---|---|---|---|
| GENE | (FPKM) | (FPKM) | s/r | p |
| PLA2G4A | 0.25 | 14.07 | 0.02 | 0.030847 |
| KCNQ1 | 0.17 | 6.33 | 0.03 | 0.046073 |
| CYB561 | 1.03 | 17.00 | 0.06 | 0.025966 |
| RAB32 | 0.77 | 10.99 | 0.07 | 0.042962 |
| LTBR | 1.27 | 17.65 | 0.07 | 0.033084 |
| MGST1 | 3.63 | 34.14 | 0.11 | 0.015628 |
| JAG1 | 3.13 | 22.38 | 0.14 | 0.032496 |
| SPART | 6.41 | 43.25 | 0.15 | 0.008230 |
| FUCA2 | 4.77 | 31.99 | 0.15 | 0.005724 |
| PRRG4 | 1.31 | 8.73 | 0.15 | 0.021411 |
| FAM83H | 2.44 | 15.09 | 0.16 | 0.047964 |
| IMPACT | 3.95 | 22.80 | 0.17 | 0.006000 |
| TMEM173 | 13.25 | 76.19 | 0.17 | 0.019256 |
| EHHADH | 1.01 | 5.04 | 0.20 | 0.034951 |
| ANTXR2 | 8.58 | 42.47 | 0.20 | 0.029025 |
| STOM | 16.21 | 75.29 | 0.22 | 0.049030 |
| FNDC3B | 18.02 | 81.15 | 0.22 | 0.021505 |
| DCBLD1 | 3.30 | 14.44 | 0.23 | 0.032619 |
| WDFY3 | 6.75 | 26.96 | 0.25 | 0.028269 |
| MIR4435- | 5.23 | 20.64 | 0.25 | 0.044500 |
| 2HG | | | | |
| PSEN2 | 3.56 | 13.77 | 0.26 | 0.017669 |
| UBR5-AS1 | 1.58 | 6.06 | 0.26 | 0.032505 |
| GALNT11 | 3.19 | 12.21 | 0.26 | 0.034382 |
| LGALS1 | 49.46 | 183.56 | 0.27 | 0.033204 |
| ERVK13-1 | 3.28 | 11.94 | 0.27 | 0.028320 |
| PLSCR1 | 13.07 | 46.21 | 0.28 | 0.043905 |
| SELENOT | 16.96 | 56.37 | 0.30 | 0.022060 |
| GALC | 7.60 | 24.38 | 0.31 | 0.036132 |
| FZD6 | 9.17 | 28.81 | 0.32 | 0.027781 |
| NEK3 | 5.05 | 15.84 | 0.32 | 0.007786 |
| TMBIM1 | 19.55 | 60.00 | 0.33 | 0.015599 |
| LINC01814 | 2.00 | 5.85 | 0.34 | 0.015652 |
| LINC01873 | 2.05 | 5.89 | 0.35 | 0.046414 |
| TRIQK | 6.03 | 17.19 | 0.35 | 0.041219 |
| BIN2 | 18.81 | 53.52 | 0.35 | 0.044227 |
| HCG11 | 6.16 | 17.31 | 0.36 | 0.021129 |
| AP1S3 | 4.08 | 11.39 | 0.36 | 0.032796 |
| CYTOR | 5.58 | 15.53 | 0.36 | 0.045003 |
| SPECC1 | 9.40 | 25.45 | 0.37 | 0.014920 |
| CFLAR | 59.96 | 160.93 | 0.37 | 0.014978 |
| CICP14 | 4.23 | 11.07 | 0.38 | 0.034708 |
| PLD3 | 38.71 | 100.79 | 0.38 | 0.023164 |
| CD151 | 21.27 | 54.32 | 0.39 | 0.018418 |
| TTC38 | 7.81 | 19.88 | 0.39 | 0.024845 |
| OXR1 | 22.60 | 57.05 | 0.40 | 0.010473 |
| ACSL1 | 35.95 | 90.39 | 0.40 | 0.038277 |
| INPP1 | 4.44 | 11.03 | 0.40 | 0.027590 |
| PLOD3 | 21.22 | 52.26 | 0.41 | 0.010786 |
| DHX32 | 6.81 | 16.67 | 0.41 | 0.045656 |
| BOLA2- | 2.75 | 6.67 | 0.41 | 0.029782 |
| SMG1P6 | | | | |
| SIL1 | 12.57 | 30.50 | 0.41 | 0.001451 |
| ST3GAL1 | 39.72 | 96.02 | 0.41 | 0.042088 |
| TMEM150A | 2.27 | 5.49 | 0.41 | 0.030789 |
| PLBD2 | 19.50 | 46.97 | 0.42 | 0.005737 |
| PDIA5 | 11.22 | 26.88 | 0.42 | 0.008020 |
| ZC3HAV1L | 4.47 | 10.71 | 0.42 | 0.038678 |
| LINC01410 | 6.62 | 15.65 | 0.42 | 0.039395 |
| SLC38A5 | 32.82 | 76.81 | 0.43 | 0.035541 |
| MYDGF | 35.01 | 81.74 | 0.43 | 0.010214 |
| BST2 | 51.61 | 119.74 | 0.43 | 0.015335 |
| IL15RA | 6.52 | 15.07 | 0.43 | 0.034222 |
| MFSD1 | 30.56 | 69.17 | 0.44 | 0.047859 |
| EXOC6B | 5.17 | 11.52 | 0.45 | 0.026993 |
| FAH | 14.82 | 32.95 | 0.45 | 0.045085 |
| SLC19A2 | 9.91 | 22.03 | 0.45 | 0.037758 |
| HSP90B1 | 404.12 | 893.99 | 0.45 | 0.012725 |
| TTC12 | 2.82 | 6.23 | 0.45 | 0.048482 |
| AGAP9 | 4.83 | 10.50 | 0.46 | 0.024958 |
| MLKL | 8.31 | 17.94 | 0.46 | 0.023925 |
| PLEC | 93.74 | 201.55 | 0.47 | 0.039854 |
| SMPD1 | 6.51 | 14.00 | 0.47 | 0.043341 |
| P4HB | 353.59 | 754.87 | 0.47 | 0.024955 |
| PLOD1 | 31.71 | 66.39 | 0.48 | 0.044380 |
| GALNT10 | 28.56 | 59.73 | 0.48 | 0.045596 |
| ACOT2 | 6.10 | 12.75 | 0.48 | 0.019467 |
| NBPF14 | 6.13 | 12.75 | 0.48 | 0.031026 |
| HSPA5 | 301.00 | 621.63 | 0.48 | 0.006884 |
| ZBTB7B | 28.29 | 58.11 | 0.49 | 0.018458 |
| NBPF10 | 8.89 | 18.18 | 0.49 | 0.025776 |
| CD63 | 66.51 | 135.87 | 0.49 | 0.014704 |
| CREB3L2 | 49.43 | 100.12 | 0.49 | 0.045806 |
| RAB4A | 13.57 | 27.42 | 0.49 | 0.024236 |
| CTSA | 39.28 | 78.77 | 0.50 | 0.028156 |
| MFSD10 | 34.76 | 69.66 | 0.50 | 0.037063 |
| C4orf36 | 3.26 | 6.53 | 0.50 | 0.001180 |

Among the genes exhibiting a sensitivity to TSA, whether upregulated or downregulated, are those associated with B-cell maturation. The CD24 gene, for example, codes for a small glycosylphosophatidylinositol (GPI)-linked glycoprotein expressed at the surface of most B lymphocyte precursors, neutrophils, and epithelial cells. It is also frequently found to be highly expressed in various hematological and solid neoplasms. CD24 also plays a role in the activation and differentiation of such cells. Bone marrow samples lacking CD24 resulted in decreased numbers of both pre-B and immature B-cell populations. CD24 expression is increased more than three-fold among cell line models sensitive to TSA treatment.

In addition, the IKZF2 gene, coding for a transcription factor regulating lymphocyte development and quiescence, is increased among cell lines sensitive to TSA. IKZF2 is frequently deleted in hypodiploid acute B lymphoblastic leukemia (B-ALL). This suggests that IKZF2 has a role as a tumor suppressor and in regulating the balance of self-renewal and differentiation in leukemic stem cells.

Embodiments of the invention include the inhibition of tumor growth by administering TSA to cancer patients exhibiting increased expression of one or more of the genes listed in Table 1 and/or decreased expression of one or more of the genes listed in Table 2. Accordingly, methods according to the invention include determining or having determined, from a biological sample obtained from a patient, whether the individual exhibits greater-than-normal expression of one or more gene listed in Table 1 or exhibits lesser-than-normal expression of one or more gene listed in Table 2.

As used herein, greater-than-normal expression shall mean expression greater than a recognized range of normal expression expected for a particular cell type or expression greater than a point midway between the expression of the TSA-resistant cell line and the TSA-sensitive cell line in Table 1. Similarly, as used herein, lesser-than-expression shall mean expression less than a recognized range of normal expression for a particular cell type or expression less than a point midway between the expression of the TSA-resistant cell line and the TSA-sensitive cell line in Table 2.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. In a method of administering trichostatin A (TSA) to a cancer patient, the improvement comprising:
selecting as said patient an individual identified as;
overexpressing at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP; or
underexpressing at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36.

2. The improvement of claim 1, wherein the individual is identified as overexpressing the CD24 gene.

3. The improvement of claim 1, wherein the individual is identified as overexpressing the IKZF2 gene.

4. (canceled)

5. A method of inhibiting tumor growth in a cancer patient, the method comprising:
determining or having determined that the patient exhibits:
increased expression of at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP; or
decreased expression of at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36; and
administering to the patient trichostatin A (TSA) in an amount effective to inhibit tumor growth.

6. The method of claim 5, wherein the patient is determined to overexpress the CD24 gene.

7. The method of claim 5, wherein the patient is determined to overexpress the IKZF2 gene.

8. A method of enhancing a tumor suppression therapy in which a cancer patient is administered a tumor suppression composition, the method comprising:
determining or having determined that the patient exhibits:
increased expression of at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP; or
decreased expression of at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36; and
administering to the patient trichostatin A (TSA) in an amount effective to inhibit tumor growth.

9. The method of claim 8, wherein the patient is determined to overexpress the CD24 gene.

10. The method of claim 8, wherein the patient is determined to overexpress the IKZF2 gene.

11. The improvement of claim 1, wherein the improvement comprises selecting as said patient an individual identified as:
overexpressing at least one gene selected from a group consisting of: TLE4, SMO, GPC2, SNTA1, YPEL1, PLEKHA2, GPR137C, CD72, ZFP36L1, PAX5, ZCCHC7, ADARB1, SMIM14, SNX22, AFF2, GJC1, RIPOR2, ARID5B, RAB9B, MIR570HG, CD79B, IKZF2, ZNF318, EBF1, SOX4, TRIB2, GRAP, TTC24, RASGRP1, MIR5195, FAM81A, IGHV5-78, LRMP, MS4A1, GPR146, CHI3L2, ZNF608, CDC25B, C12orf77, GPR18, PCDH12, SCML4, CD24, INA, TUNAR, FAM241B, TCL1B, IGLV1-44, and ENPEP.

12. The improvement of claim 1, wherein the improvement comprises selecting as said patient an individual identified as underexpressing at least one gene selected from a group consisting of: PLA2G4A, KCNQ1, CYB561, RAB32, LTBR, MGST1, JAG1, SPART, FUCA2, PRRG4, FAM83H, IMPACT, TMEM173, EHHADH, ANTXR2, STOM, FNDC3B, DCBLD1, WDFY3, MIR4435-2HG, PSEN2, UBR5-AS1, GALNT11, LGALS1, ERVK13-1, PLSCR1, SELENOT, GALC, FZD6, NEK3, TMBIM1, LINC01814, LINC01873, TRIQK, BIN2, HCG11, AP1S3, CYTOR, SPECC1, CFLAR, CICP14, PLD3, CD151, TTC38, OXR1, ACSL1, INPP1, PLOD3, DHX32, BOLA2-SMG1P6, SIL1, ST3GAL1, TMEM150A, PLBD2, PDIA5, ZC3HAV1L, LINC01410, SLC38A5, MYDGF, BST2, IL15RA, MFSD1, EXOC6B, FAH, SLC19A2, HSP90B1, TTC12, AGAP9, MLKL, PLEC, SMPD1, P4HB, PLOD1, GALNT10, ACOT2, NBPF14, HSPA5, ZBTB7B, NBPF10, CD63, CREB3L2, RAB4A, CTSA, MFSD10, and C4orf36.
